(19) United States
(12) Patent Application Publication
(10) Pub. No.: US 2016/0123957 A1
(43) Pub. Date: May 5, 2016

(54) Visualization of P-TEFb by Fluorescence Complementation

(71) Applicant: The Regents of the University of California, Oakland, CA (US)

(72) Inventors: Boris Peterlin, San Francisco, CA (US); Koh Fujinaga, San Francisco, CA (US)

(21) Appl. No.: 14/932,611

(22) Filed: Nov. 4, 2015

Related U.S. Application Data
(60) Provisional application No. 62/075,227, filed on Nov. 4, 2014.

(51) Int. Cl. G01N 33/50; C12N 9/12

(52) U.S. Cl. CPC G01N 33/502; C12N 9/12; C12Y 207/11012; G01N 2333/912; G01N 2800/52; G01N 2500/10

(57) ABSTRACT

Provided herein is a novel assay for the quantification of P-TEFb activation in living cells. The invention, in one embodiment, comprises cells which express P-TEFb and a P-TEFb-phosphorylated or P-TEFb-binding species, for example the C terminal domain of RNA polymerase II. Each of the P-TEFb and P-TEFb-phosphorylated or P-TEFb-binding species comprises a complementary signal moiety, for example complementary fragments of a fluorescent protein, such that when the P-TEFb interacts with the P-TEFb-phosphorylated or P-TEFb-binding species, the signal moieties are in sufficiently close proximity to generate a detectable signal.

### CROSS-REFERENCE TO RELATED APPLICATIONS

This application claims the benefit of priority to U.S. Provisional Application Ser. No. 62/075,227 entitled “Visualization of P-TEFb by Fluorescence Complementation,” filed Nov. 4, 2014, the contents which are hereby incorporated by reference.

### STATEMENT REGARDING FEDERALLY SPONSORED RESEARCH OR DEVELOPMENT

This invention was made with government support under grant nos. U19 AI076113, P50 GM082250, R01 AI049104 and GM035500 awarded by the National Institutes of Health. The government has certain rights in the invention.

### REFERENCE TO SEQUENCE LISTING, A TABLE, OR A COMPUTER PROGRAM LISTING COMPACT DISK APPENDIX

This application is submitted with a computer readable sequence listing, submitted herewith via EFS as the ASCII text file named: “UCSF014NP_SL.txt”, file size approximately 5,185 bytes, created on Nov. 4, 2015 and hereby incorporated by reference in its entirety.

### BACKGROUND OF THE INVENTION

P-TEFb is a master regulator of transcriptional elongation, a critical step to determine cell growth, differentiation and apoptosis. Much of the cellular P-TEFb exists in an inactive form, bound by HEXIM1 in the 7SK snRNP complex. Various external stimuli or internal cell processes cause the release of P-TEFb from the 7SK snRNP complex. This liberated P-TEFb is the active form, and it promotes transcription via phosphorylation of the C-terminal domain (CTD) of RNA polymerase II and negative transcription elongation factors such as NELF and DSIF. The resulting activation of transcription is a key initiator of various processes, implicated in growth, cell division, inflammation, and in pathologies such as cancer or HIV replication. Thus, the equilibrium between active and inactive states of P-TEFb is an important regulatory control point in many biological processes.

Unfortunately, there are no facile assays to assess and monitor P-TEFb equilibrium and activity in living cells. Current assays are laborious and inaccurate and do not allow P-TEFb monitoring in living, intact cells. Accordingly, there is a need in the art for an effective assay that allows observation of P-TEFb activity in living cells.

### SUMMARY OF THE INVENTION

The inventors of the present disclosure have advantageously invented a novel assay for the visualization of P-TEFb by fluorescence complementation or related methods. In one embodiment, the present invention is an adaptation of the bi-molecular fluorescence complementation (BiFC) assay technology, as known in the art, adapted in the novel context of monitoring P-TEFb activity. The invention allows for the monitoring of P-TEFb release in living cells with great accuracy, and enables the observation of the effect of various treatments on P-TEFb dynamics.

### BRIEF DESCRIPTION OF THE DRAWINGS

FIGS. 1A, 1B, and 1C. FIG. 1A depicts a control construct comprising a YFP fluorophore fragment (YC) and cMyc epitope tag (m). FIG. 1B depicts a P-TEFb construct of the invention, comprising cMyc epitope tag (m), a C-terminal YFP fluorophore fragment (YC), a Cdk9 sequence (Cd9k), and a CycT1 (CycT1) sequence. FIG. 1C depicts a CTD construct of the invention, comprising a cMyc epitope tag (m), an N-terminal YFP fluorophore fragment (YN), a nuclear localization signal, (NLS) and a series of 52 CTD heptapeptide repeats (SEQ ID NO: 7).

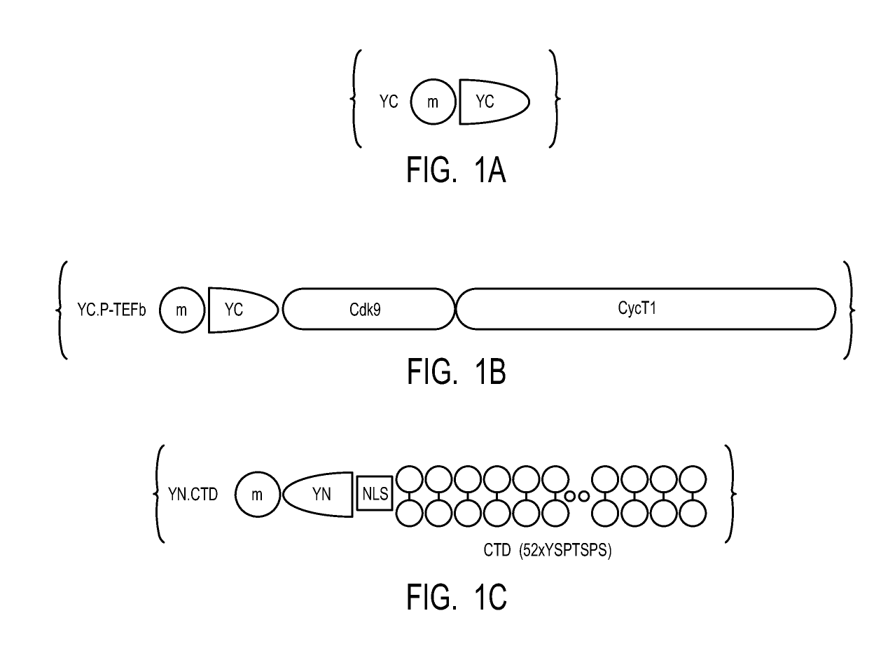

FIG. 2. FIG. 2. depicts the interaction of the elements of the invention, wherein a P-TEFb protein (P-TEFb) labeled with a C-terminal YFP fluorophore fragment (YC) is incorporated into the 7SK snRNP complex, comprising HEXIM1, McPCE, and LaRP7. The YC labeled P-TEFb is liberated, becoming free P-TEFb as a result of a P-TEFb releasing stimulus. The released YC-labeled P-TEFb then associates with a CTD construct, comprising a CTD domain (CTD) and an N-terminal YFP fluorophore fragment which complements the YC fluorophore fragment to produce signal. The resulting complex of PTEF-b, CTD and their associated fluorophores fragments (YC and YN) creates a complete and functional fluorophore (YFP) that is capable of detection.

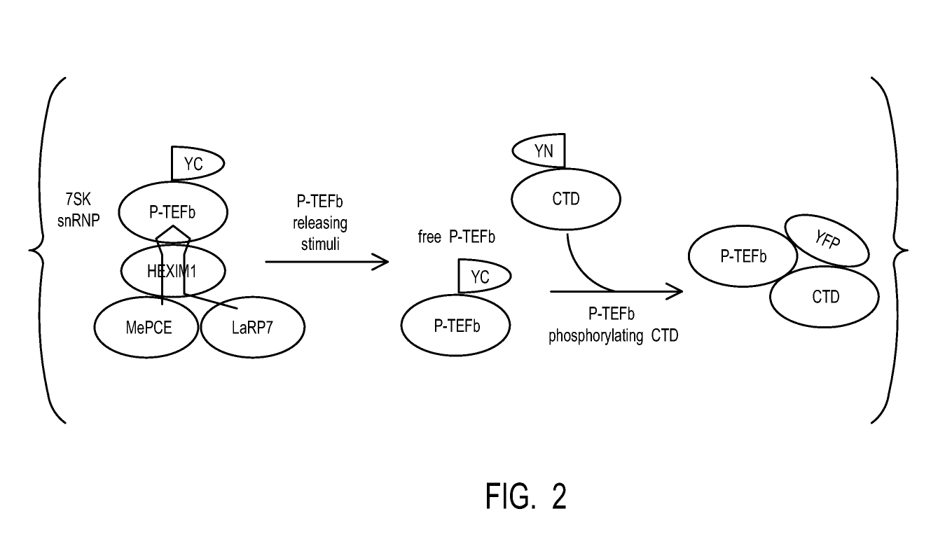

### DETAILED DESCRIPTION OF THE INVENTION

The invention encompasses methods of detecting P-TEFb activation and associated genetic constructs and biological assays. The basic operation of the invention encompasses the use of two complementary constructs. The first construct will be referred to herein as the “P-TEFb construct” and it comprises a P-TEFb protein or biologically active portion thereof which is labeled with a first moiety of a signal pair. The second construct, referred to herein as the “P-TEFb target construct” comprises a P-TEFb activated species and the second moiety of the signal pair. Both constructs are expressed in a living cell. Some portion of the expressed P-TEFb construct is incorporated into native 7SK small nuclear ribonucleoprotein (7SK snRNP) complexes in the cell, representing the sequestered or inactive form of P-TEFb. Upon a stimulus which releases P-TEFb from the 7SK snRNP, the liberated P-TEFb construct will interact with the P-TEFb target construct. The close physical association of the P-TEFb construct and the P-TEFb target construct puts the two members of the signal pair in proximity such that it becomes detectable.

Signal Pairs.

The invention utilizes two binding partners, each binding partner comprising a complementary signal moiety which, when the binding partners interact, brings the two complementary signal moieties into proximity and creates a detectable signal. The amount of signal observed is proportional to the degree of binding partner interaction. In one embodiment, the invention comprises a bimolecular fluorescence complementation assay (BiFC), wherein the two members of the signal pair are two fragments of a fluorescent protein, each fragment being undetectable or minimally detectable by itself, and wherein when the two fragments of the fluorescent protein are brought in proximity, they reconstitute a detectable fluorescent protein. The fluorescent proteins of the invention comprise any fluorescent protein known in the art which is capable of use in a BiFC system. Exemplary fluorescent proteins include yellow fluorescent protein (YFP). For example, the signal pair may comprise a first polypeptide comprising amino acids 1-154 of YFP, referred to as “YN” because it is the N-terminal fragment of YFP and a second polypeptide comprising amino acids 155-238 of YFP, referred to as YC because it is the C-terminal fragment of YFP. In another embodiment, the label pair may comprise a first polypeptide comprising amino acids 1-158 of Venus fluorescent protein, referred to as “VN,” and a second polypeptide comprising amino acids 159-239 of Venus fluorescent protein, referred to as “VC.” In another embodiment, the label pair may comprise VN and YC. Any other fluorescent protein known in the art that can be adapted for use in BiFC assays may be used, for example, YFP, YFP variants such as Venus, GFP, and other proteins, for example as described in: Kerppola, T. K., 2009, Visualization of molecular interactions using bimolecular fluorescence complementation analysis, characteristics of protein fragment complementation, Che Soc. Rev. 38: 2876-86; Kodama and Hu, 2012, “Bimolecular fluorescence complementation assay (BiFC): a five year update and future perspectives, Biotechniques 53: 285-98; and Miller et al., 2015, “Bimolecular Fluorescence Complementation (BiFC) Analysis: Advances and Recent Applications for Genome-Wide Interaction Studies,” J. Mol. Biol. 427:2039-55.

In another embodiment, the signal pair comprises a FRET pair of proteins, wherein quenching of one fluorophore of the FRET pair occurs when the two proteins of the FRET pair are in proximity, as known in the art. Exemplary FRET pairs include cyan fluorescent protein and YFP, as known in the art. FRET detection is performed as known in the art.

The signal pair may comprise any other pair of moieties, which, when in proximity, produce a detectable signal that indicates the two moieties are in proximity. For example, yeast two hybrid systems, as known in the art, may be employed in practice of the invention.

Polypeptide Constructs of the Invention.

Polypeptide construct, as used herein, means a polypeptide, such as a protein, protein fragment, protein sequence, or chimeric protein. The invention encompasses two separate polypeptide constructs. The polypeptide constructs are expressed in a cell of a target species. The polypeptide constructs, together, are functional in the target species, meaning they will produce detectable signals proportional to free P-TEFb concentrations in the cell. In some embodiments, the biological moieties of the polypeptide constructs are from the target species, ensuring compatibility with the biological environment in which they are expressed. In other embodiments, the biological moieties of the polypeptide constructs are from a different species than the target species, but which are functional in the target species. In another embodiment, the polypeptide constructs are artificial, consensus, or optimized sequences which can function in the target species.

Likewise, the signal moieties, e.g. fluorophore sequences, will be selected to be expressed, translated, and functional in the target organism, as known in the art.

The polypeptide constructs of the invention may comprise sequences derived from or functional in any eukaryotic species, including humans, mice, rats, dogs, cats, non-human primates, yeast, nematodes, and others.

P-TEFb Polypeptide Construct.

The P-TEFb polypeptide construct comprises a P-TEFb moiety, which comprises P-TEFb, or biologically active (e.g. having at least one ligand binding ability or physiological action of native P-TEFb) fragments thereof. The P-TEFb moiety must retain the ability to be sequestered within the 7SK snRNP complex and the ability to bind or otherwise interact with one or more P-TEFb target species. In one embodiment, the P-TEFb moiety is the entire P-TEFb protein. P-TEFb protein is a heterodimeric protein made of two subunits, a Cdk9 subunit and a clyclin T1 (CycT1), cyclin T2a (CycT2a) or cyclin T2b (CycT2b) subunit. P-TEFb protein or simply P-TEFb, as used herein, will refer to any combination of CDk9 and one of CycT1, CycT2a or CycT2b sequences, which such combination retains at least one biological function or ligand binding ability of native PTEF-b. In another embodiment, the P-TEFb moiety comprises substantially the whole P-TEFb protein, for example 80, 85, 90, 95, or 99% of a P-TEFb protein, including truncated versions of known P-TEFb variants, P-TEFb, or P-TEFb analogs comprising amino acid substitutions, additions, or deletions. In another embodiment, the P-TEFb moiety comprises a Cdk9 subunit of P-TEFb, or functional portions thereof and the second subunit comprising CycT1, CycT2a, or CycT2b sequences is omitted. In one embodiment, the Cdk9 sequence is the human CDk9 subunit sequence. In another embodiment, the P-TEFb moiety comprises a CycT1 subunit from P-TEFb, or functional portions thereof and the Cdk9 subunit is omitted. In one embodiment, the CycT1 sequence is the human CycT1. In another embodiment, the P-TEFb moiety comprises a Cdk9 domain and a CycT1 subunit.

Because biologically relevant P-TEFb activity is primarily situated in the nucleus, it is important the P-TEFb polypeptide construct localize to the nucleus when expressed in the target cell. If the P-TEFb moiety comprises an intact CycT1 domain, this domain contains a strong nuclear localization signal. If the P-TEFb fragment comprises a sequence lacking a functional CycT1 nuclear localization signal, then a nuclear localization signal, as known in the art, will need to be included in the P-TEFb construct.

The P-TEFb construct further comprises one signal moiety of a signal pair, for example a fluorophore fragment, as described above. The signal moiety may be joined to the P-TEFb fragment at either the carboxy or amino terminus of the P-TEFb fragment. As the C-terminal portion of P-TEFb is the domain which interacts with the CTD to facilitate its phosphorylation, in some embodiments it is preferred that the fluorophore fragment be joined to the P-TEFb fragment at its amino terminal end to avoid steric interference with the CTD binding. An exemplary fluorophore fragment is the carboxy-terminal portion of yellow fluorescent protein, as known in the art.

The P-TEFb construct may optionally include a linker sequence connecting the fluorophores (or other label moiety) to its associated biologically active fragment. The linker is a flexible chain of amino acids which allows the fluorophore fragment some freedom of movement, allowing it to more efficiently find and conjugate to its complementary fluorophore fragment. Linker sequences are known in the art, for example (in single letter amino acid code), sequences such as RSIAT (SEQ ID NO: 1), RPACKIPNDLKQKVMNH (SEQ ID NO: 2), AAANSSIDLISVPVDSR (SEQ ID NO: 3), or VFGGTGGGSGGGSGGGSGGGTSGSEFP (SEQ ID NO: 6). In some embodiments, the linker sequence is not used.

The P-TEFb Target Polypeptide Construct.

The P-TEFb target polypeptide construct comprises a moiety that binds to, associates with or otherwise interacts with free P-TEFb. Exemplary P-TEFb target moieties include the C-terminal domain of RNA polymerase II. Other P-TEFb target species are known in the art and include the 5,6-dichloro-1-β-D-ribofuranosylbenzimidazole sensitivity-inducing factor (DSIF) and negative elongation factor (NELF). In one embodiment, the P-TEFb activated moiety is a CTD construct. The CTD construct is a protein sequence based upon the carboxy terminal portion of the RNA polymerase II large subunit, consisting of multiple heptapeptide repeats of the consensus sequence YSPTSPS (SEQ ID NO: 4). The number of repeats varies from 26 or 27 in yeast to 52 in mammals, for example in one embodiment the CTD moiety is a 52X repeat of SEQ ID NO: 4 (SEQ ID NO: 7). The carboxy terminal fragment of the invention may comprise the precise number of heptapeptide repeats found in the target species. In an alternative embodiment, the CTD domain comprises a truncated version (containing less heptapeptides) or an elongated version (containing more heptapeptides) than the CTD domain found in the target species.

In the CTD construct, the label moiety, e.g. fluorophore fragment, may be on either the carboxy- or amino-terminus of the protein. A preferred implementation is to have the signal, e.g. fluorophores, fragment on the amino-terminal side of the CTD domain.

The P-TEFb target moiety, for example a CTD construct, should generally include a nuclear localization sequence so that it will translocate to the nuclear region where most biologically relevant P-TEFb activity occurs. For example, a nuclear localization sequence which also doubles as a flexible linker may be used, for example the tether sequence: KRPAATKKAGQAKKKK (SEQ ID NO: 5). In another embodiment, the CTD construct comprises separate linker and nuclear localization sequences.

Accessory Features.

The constructs of the invention may further comprise accessory features or domains. For example, an antibody epitope may be conjugated to the n-terminal or c-terminal end of each construct to facilitate imaging of the constructs in vivo. An exemplary antibody epitope is the cMyc epitope sequence, as known in the art, for example, being located at the n-terminal end of each construct.

Gene Constructs.

The scope of the invention encompasses the chimeric P-TEFb and P-TEFb target protein polypeptide constructs of the invention, as described above. The scope of the invention further encompasses nucleic acid sequences which encode such polypeptide constructs. For example, the nucleic acid constructs of the invention may comprise cloning vectors, expression vectors, transformation vectors, and other nucleic acid constructs. For example, in one embodiment, the invention comprises a kit, comprising a polynucleotide which codes for a P-TEFb construct and a polynucleotide which codes for a P-TEFb target construct.

The nucleic acid sequences of the invention may further comprise promoters or other regulatory elements for desired expression characteristics. For example, the constructs may be constitutively expressed, transiently expressed, or may be inducibly expressed by use of inducible promoters and appropriate stimuli. Additionally, the nucleic acid sequences may be expressed under the control of developmental promoters in order to observe cellular responses to developmental events.

Generally, it will be preferred that the signal moieties of the signal pair are peptides that can be expressed with the P-TEFb or P-TEFb-activated moieties, such that each construct can be expressed as a single chimeric protein. However, it will be understood that signal moieties and other accessory features of the construct can be conjugated, chemically bound, or otherwise attached to the biologically functional moieties of the constructs post-translationally.

Transformation of Target Cells.

The nucleic acid sequences of the invention may be introduced into the target biological system by any transformation technology known in the art. For example, transient expression systems may be used, or the nucleic acid sequences may be stably transformed into the target system. The target cells may comprise whole organisms, isolated tissues, cultured cells, explanted cells, or single cells.

In one embodiment, the invention comprises a cell or whole organism which expresses one or both of a P-TEFb construct and a P-TEFb target construct.

P-TEFb Assays of the Invention.

The methods of the invention are carried out as follows. In a transformed biological system where the two polypeptide constructs of the invention have been effectively expressed, a significant portion of the expressed P-TEFb polypeptide construct will be sequestered in the 7SK snRNP complex, as is native P-TEFb. Background signal from the signal pair can be measured in such cells to establish the baseline level of signal in an unperturbed system.

Next, a stimulus is applied to the biological system. The stimulus can be any stimulus, for example a chemical, electrical, physical, or other stimulus. For example, in one embodiment the stimulus is the administration to the biological system of a chemical species (e.g. a drug or putative drug) or other biologically active species (e.g. a protein, including growth factors, etc.).

Upon a cellular stimuli or event that triggers the release of active P-TEFb, some portion of the P-TEFb construct sequestered in the 7SK snRNP complex will be liberated and will interact with the P-TEFb target constructs present in the cells such that the complementary signal fragments are in sufficient proximity to generate a detectable signal. This signal will be proportional to the degree of P-TEFb release occurring in the cell, and thus the invention provides a means of quantitatively monitoring P-TEFb activity in living cells.

The signal can be measured using devices and methods appropriate for the particular signal moieties in use. For example, where the signal moieties are fluorescent protein fragments, fluorescence may be imaged using excitation and microscopy systems appropriate for the selected fluorophores. The measurable signal in treated cells can be compared to that in a control group (e.g. an untreated or sham treated group of like cells) to qualitatively and quantitatively assess the effects of the treatment on P-TEFb activity.

In one embodiment, a fluorescently activated cell sorting (FACS) process is utilized to sort and quantify cells which have a fluorescent signal induced by P-TEFb release. For example, gating protocols can be set to isolate cells which express the signal of the reconstituted fluorophore or other signal moiety, such gating set at signal thresholds indicative of stimulus induced P-TEFb. In a treated group of cells wherein a stimulus is applied, the number of fluorescent cells expressing the reconstituted signal moiety above the threshold can be quantified by FACS and the effect of the treatment stimulus on P-TEFb activity can be assessed by comparing the number of fluorescent cells meeting the threshold in the treated sample to that observed in a control group.

Detection of signal can be accomplished at varying time intervals. For example, observations made 1 to 360 minutes after application of the stimulus can be used, for example, measurements at 60-75 minutes after the stimulus is applied.

The system of the invention may be used in various contexts. For example, cellular response to various stimuli may be monitored to observe P-TEFb activity and/or localization of such activity. In one embodiment, the invention comprises a method of assessing a treatment's effect on P-TEFb activity in the target cells. In one embodiment, the invention comprises a screening protocol for the identification of P-TEFb modulator agents, such as effectors or inhibitors, from a pool of putative modulators, e.g. in a high throughput screen. The target cells are exposed to a putative P-TEFb modulator and the resulting signal, e.g. fluorescence of the reconstituted fluorophores, is measured. Where a substantial increase of fluorescence is observed, the putative modulator is an agonist of P-TEFb release, and likewise, where an expected fluorescence signal is attenuated or obliterated, the putative modulator is identified as an antagonist of P-TEFb release.

Increased P-TEFb activity and the subsequent synthesis of its inhibitor HEXIM1 represent critical common steps for many anti-cancer and anti-inflammatory drugs. Importantly, it is this reassembly of the 7SK snRNP following the increased synthesis of HEXIM1 that causes proliferating cells to differentiate and rapidly dividing cells to arrest and undergo apoptosis. Thus, the assay of the invention may advantageously be used to reveal compounds that can be used against inflammation and cancer and also for HIV reactivation.

Personalized Medicine Applications.

In another implementation, the invention may be used in a personalized medicine context. Cells directly extracted from a patient or primary cells derived from a patient may be transformed to express the constructs of the invention, and the cellular response to various P-TEFb modulators may then be observed, in order to determine whether the patient is amenable to or incompatible with various P-TEFb modulating treatments.

### EXAMPLES

### Example 1

### Demonstration of the P-TEFb Assay in Cultured Cells

Sequences encoding amino acids 1-154 (YN) and 155-238 (YC) of YFP were amplified and cloned in mammalian expression plasmids. The YN.CTD chimera was constructed by inserting DNA fragments from a 52X heptapeptide (52 X SEQ ID NO: 4 (SEQ ID NO: 7)) CTD coding plasmid with a linker sequence encoding a nuclear localization signal, SEQ ID NO: 5. The YC.PTEFb chimera was constructed by inserting DNA fragments corresponding to P-TEFb into the YC plasmid, with Cdk9 and CycT1 sequences genetically fused to express as a single chimeric polypeptide, that can form a functional P-TEFb molecule. A polynucleotide coding for the linker of SEQ ID NO: 6 was optionally utilized in the constructs, between P-TEFb or CTD sequences and their associated fluorophores fragments. All proteins contained an anti-c-Myc antibody epitope tag at the N termini, and their expression in cells was confirmed by Western blotting using an anti-c-Myc antibody.

HeLa or HEK293 cells growing in log phase were transfected with plasmid DNA encoding YN.CTD and YC.P-TEFb fusion proteins by use of a lipofection agent. Incorporation of YC.P-TEFb into the 7SK snRNP was confirmed by immunoprecipitation and Western blot analysis with antibodies to the components of the 7SK snRNP. SAHA treatment reduced the amounts of YC.P-TEFb and endogenous CycT1 proteins in 7SK snRNP fractions and increased the levels of free P-TEFb.

When cells are treated with P-TEFb-releasing agents, P-TEFb dissociates rapidly from the 7SK snRNP, which liberates its kinase activity. Depending on the stimulus, this process occurs within a few minutes to 1 h.

The BiFC assay was used to detect interactions between released free P-TEFb and its natural substrate, RNA polymerase II CTD. When YC.P-TEFb was coexpressed with YN.CTD in HEK293 cells under normal culture conditions, very few weakly YFP-positive cells were detected. When cells were stimulated with known P-TEFb-releasing agents, such as HMBA and SAHA, the number of YFP-positive increased significantly. Because the emission peak of YFP (527 nm) is similar to that of GFP (509 nm), the BiFC-positive cells appeared green. In sharp contrast, when cells were treated with tubastatin A, a potent selective HDAC inhibitor that has no effect on P-TEFb release, the number of YFP positive cells did not increase. In addition, YN.CTD did not produce any fluorescence when coexpressed with YC as the negative control, confirming the specificity of the BiFC signal. YC.P-TEFb was employed to monitor P-TEFb activation by BiFC in cells. Observation of YFP positive cells at a higher magnification revealed that the fluorescent signals accumulated in punctate nuclear structures in a speckle pattern, which is consistent with previous observations of P-TEFb nuclear localization.

A time-dependent increase in BiFC signals was monitored by time-lapse fluorescence microscopic analysis. HEK293 cells expressing YC.P-TEFb and YN.CTD were treated with 5 micromolar SAHA, and fluorescent images were taken every 3 min. BiFC signals were detected by 30 min after the addition of SAHA and reached a peak at a time point between 75 and 90 min. At later times, more intense and punctate nuclear staining reflected the accumulation of active P-TEFb in nuclear speckles. This finding is consistent with previous observations of P-TEFb release by SAHA with a consideration of the time required for maturation of the fluorophore. Similar results were obtained with other P-TEFb-releasing agents, such as HMBA and JQ1, ST80, and bryostatin-1. AzaC also released P-TEFb in the assay. It has been demonstrated previously that AzaC activates HIV transcription potently in latently infected cells and that many agents that activate HIV transcription in latently infected cells also release P-TEFb from the 7SK snRNP. Therefore, it was not surprising that AzaC also released P-TEFb from the 7SK snRNP although the precise molecular mechanism of P-TEFb release is currently unknown.

It was also examined whether YC fused with each component (CDK9 or CycT1) of P-TEFb produced BiFC signals with YN.CTD. Both produced BiFC signals, but they were less efficient than those observed with YC.P-TEFb and YN.CTD.

Assaying titrations in different cell lines, 1:5 to 1:10 ratios between plasmids encoding YC.P-TEFb and YN.CTD gave the best stimulation-dependent BiFC signals versus background fluorescence. One way to avoid such titrations in different cell lines would be to establish cells stably expressing our chimeric YC.P-TEFb and YN.CTD proteins

Because interactions between kinases and their substrate are considered to be transient and because kinases dissociate upon phosphorylating their substrates, it is somewhat surprising that a kinase (P-TEFb) and its substrate (CTD) produce such a strong BiFC signal. Nevertheless, it has been demonstrated previously that P-TEFb does form a stable complex with the CTD. This finding could be due to the large number of substrate residues, i.e. serines at position 2 in the CTD. In addition, once the BiFC fluorophore is formed, it stabilizes itself, resulting in a much slower dissociation rate. Therefore, once YC.P-TEFb binds to YN.CTD, it produces sustainable BiFC signals. These results demonstrate the first experimental system to monitor P-TEFb activation in living cells. The assay is ideally suited to monitor single agents and combinations of compounds and measure their impact on P-TEFb release in living cells.

### Example 2

### Demonstration of the P-TEFb Assay Employing Venus Fluorescent Protein in HIV-1 Post-Integration Latency Model Cell Lines

The persistence of latently infected cells in patients under combinatory antiretroviral therapy is a major hurdle to HIV-1 eradication. Strategies to purge these reservoirs are needed, and activation of viral gene expression in latently infected cells is one promising strategy. Bromodomain and Extraterminal (BET) bromodomain inhibitors (BETi) are compounds able to reactivate latent proviruses in a positive transcription elongation factor b (P-TEFb)-dependent manner. In this study, the reactivation potential of protein kinase C (PKC) agonists (prostratin, bryostatin-1 and ingenol-B), which are known to activate P-TEFb was tested, used alone or in combination with P-TEFb-releasing agents (HMBA and BETi (JQ1, I-BET, I-BET151)). Using in vitro HIV-1 post-integration latency model cell lines of T-lymphoid and myeloid lineages, it was demonstrated that PKC agonists and P-TEFb-releasing agents alone acted as potent latency-reversing agents (LRAs) and that their combinations led to synergistic activation of HIV-1 expression at the viral mRNA and protein levels.

In order to study the effect of the PKC agonist+BETi/HMBA combined treatments on P-TEFb activation in the model cells, the bimolecular fluorescence complementation (BiFC) assay of the invention was utilized, with the N-terminal region of Venus fluorescent and the C-terminal region of YFP fluorescent protein used as the signal pair. P-TEFb and the CTD were used as fusion partners of YC and VN, respectively. The results demonstrated that the number of YFP-positive cells was higher following the combined bryostatin-1+JQ1 treatment than the numbers obtained after the individual drug treatments. These data strongly indicated that combined latency reversing agent treatments led to higher activations of P-TEFb than the corresponding individual drug treatments, and that the use of such combinations presents a promising latency-reversing treatment strategy.

All patents, patent applications, and publications cited in this specification are herein incorporated by reference to the same extent as if each independent patent application, or publication was specifically and individually indicated to be incorporated by reference in its entirety. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A cell which expresses
a first polypeptide comprising a P-TEFb moiety, such P-TEFb moiety comprising a P-TEFb protein or biologically active fragment thereof, and a first signal moiety; and
a second polypeptide comprising a P-TEFb target moiety and a second signal moiety;
wherein when the first and second polypeptides are interacting, the first and second signal moieties are placed in proximity to each other, producing a detectable signal.

2. The cell of claim 1, wherein
the P-TEFb moiety comprises a P-TEFb protein.

3. The cell of claim 1, wherein
the P-TEFb moiety comprises a CDK9 subunit.

4. The cell of claim 1, wherein
the P-TEFb moiety comprises a CycT1, CycT2a, or CycT2b subunit.

5. The cell of claim 1, wherein
the P-TEFb target moiety comprises a CTD domain from RNA polymerase II.

6. The cell of claim 5, wherein
the CTD domain from RNA polymerase II comprises SEQ ID NO: 7.

7. The cell of claim 1, wherein
the first and the second polypeptide comprise a nuclear localization signal.

8. The cell of claim 1, wherein
the first and second signal moieties comprise complementary portions of a fluorescent protein.

9. The cell of claim 8, wherein
the first or second signal moiety comprises a fragment of YFP.

10. The cell of claim 8, wherein
the first or second signal moiety comprises a fragment of Venus fluorescent protein.

11. The cell of claim 1, wherein
the first polypeptides comprises a linker sequence between the P-TEFb moiety and the first signal moiety; and
the second polypeptides comprises a linker sequence between the P-TEFb target moiety and the second signal moiety.

12. A method of determining the effect of a treatment on the release of P-TEFb in a living cell, wherein the cell expresses a first polypeptide comprising a P-TEFb moiety, such P-TEFb moiety comprising a P-TEFb protein or biologically active fragment thereof, and a first signal moiety, and the cell expresses a second polypeptide comprising a P-TEFb target moiety, a second signal moiety, and a nuclear localization signal, and wherein the first and second signal moieties, when in proximity to each other, produce a detectable signal, comprising the steps of
subjecting the cell to a treatment; and
subsequently observing the amount of signal in the treated cell;
wherein, an elevated amount of signal observed in the treated cell, relative to control group cells, is indicative that the treatment increased the release of free P-TEFb.

13. The method of claim 12, wherein
the P-TEFb moiety is a P-TEFb protein and the P-TEFb targeted moiety is a CTD domain of RNA polymerase II.

14. The method of claim 12, wherein
the first and second signal moieties comprise complementary portions of a fluorescent protein.

15. The method of claim 14, wherein
the first or second signal moiety comprises a fragment of YFP.

16. The method of claim 14, wherein
the first or second signal moiety comprises a fragment of Venus fluorescent protein.

17. The method of claim 12, wherein
the treatment is the administration of a chemical composition.

18. The method of claim 17, wherein
the chemical composition is a putative P-TEFb releasing composition.

19. The method of claim 12, wherein
the measurement of signal is conducted 30-90 minutes after application of the treatment.


SEQUENCE LISTING (7 sequences)

SEQ ID NO: 1 - protein, 5 aa, Artificial Sequence
  Description of Artificial Sequence Synthetic peptide
RSIAT

SEQ ID NO: 2 - protein, 17 aa, Artificial Sequence
  Description of Artificial Sequence Synthetic peptide
RPACKIPNDLKQKVMNH

SEQ ID NO: 3 - protein, 17 aa, Artificial Sequence
  Description of Artificial Sequence Synthetic peptide
AAANSSIDLISVPVDSR

SEQ ID NO: 4 - protein, 7 aa, Artificial Sequence
  Description of Artificial Sequence Synthetic peptide
YSPTSPS

SEQ ID NO: 5 - protein, 16 aa, Artificial Sequence
  Description of Artificial Sequence Synthetic peptide
KRPAATKKAGQAKKKK

SEQ ID NO: 6 - protein, 27 aa, Artificial Sequence
  Description of Artificial Sequence Synthetic peptide
VFGGTGGGSGGGSGGGSGGGTSGSEFP

SEQ ID NO: 7 - protein, 364 aa, Artificial Sequence
  Description of Artificial Sequence Synthetic polypeptide
YSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPS